SEQUENCE LISTING (9 sequences)

SEQ ID NO: 1 - protein, 41 aa, Artificial Sequence
  synthetic peptide
HSQGTFTSDYSKYLDSRRAQDFVQWLMNTRHYLNLVTRQRY

SEQ ID NO: 2 - protein, 34 aa, Homo sapiens
IKPEAPGEDASPEELNRYYASLRHYLNLVTRQRY

SEQ ID NO: 3 - protein, 29 aa, Homo sapiens
HSQGTFTSDYSKYLDSRRAQDFVQWLMNT

SEQ ID NO: 4 - protein, 29 aa, Homo sapiens
HAEGTFTSDVSSYLEGQAAKEFIAWLVKG

SEQ ID NO: 5 - protein, 40 aa, Artificial Sequence
  synthetic peptide
HSQGTFTSDLSKQMEEEAVRLFIEWLKNRHYLNLVTRQRY

SEQ ID NO: 6 - protein, 46 aa, Artificial Sequence
  synthetic peptide
HSQGTFTSDLSKQMEEEAVRLFIEWLKNRYYASLRHYLNLVTRQRY

SEQ ID NO: 7 - protein, 44 aa, Artificial Sequence
  synthetic peptide
HSQGTFTSDLSKQMEEEAVRLFIEWLKNGGPSRHYLNLVTRQRY

SEQ ID NO: 8 - protein, 50 aa, Artificial Sequence
  synthetic peptide
HSQGTFTSDLSKQMEEEAVRLFIEWLKNGGPSRYYASLRHYLNLVTRQRY

SEQ ID NO: 9 - protein, 47 aa, Artificial Sequence
  synthetic peptide
HSQGTFTSDYSKYLDEEAARLFIEWLMNTRYYASLRHYLNLVTRQRY

(19) United States
(12) Patent Application Publication
(10) Pub. No.: US 2019/0211072 A1
(43) Pub. Date: Jul. 11, 2019

(54) TRI-AGONIST FOR THE GLu, GLP-1 AND NPY2 RECEPTORS

(71) Applicant: Robert P. Doyle, Manlius, NY (US)

(72) Inventor: Robert P. Doyle, Manlius, NY (US)

(73) Assignee: Syracuse University, Syracuse, NY (US)

(21) Appl. No.: 16/241,538

(22) Filed: Jan. 7, 2019

Related U.S. Application Data
(60) Provisional application No. 62/615,669, filed on Jan. 10, 2018.

(51) Int. Cl. C07K 14/605; C07K 14/575

(52) U.S. Cl. CPC C07K 14/605; A61K 38/00; C07K 2319/75; C07K 14/57545

(57) ABSTRACT

A monomeric peptide that functions as an agonist for the glucagon receptor (GluR), the glucagon-like peptide 1 receptor (GLP1-R) and neuropeptide Y2 receptor (NPY2-R). The peptide thus targets three of the receptors involved glucoregulation and appetite regulation to more efficiently and completely facilitate weight loss in, among others, type II diabetic patients while also being capable of stimulating a reduction in appetite to complement the weight loss results.

### CROSS-REFERENCE TO RELATED APPLICATIONS

The present application claims priority to U.S. Provisional App. No. 62/615,663, filed on Jan. 10, 2018.

### BACKGROUND OF THE INVENTION

### 1. Field of the Invention

The present invention relates to peptide agonists and, more particularly, to a tri-agonist for the glucagon receptor (GluR), the glucagon-like peptide 1 receptor (GLP1-R) and neuropeptide Y2 receptor (NPY2-R).

### 2. Description of the Related Art

The worldwide prevalence of obesity, diabetes, and associated metabolic complications increase the risk of cardiovascular disease and stroke, which collectively present a great threat to public health. There are, however, a number of different peptides and receptors involved in the glucoregulation and appetite regulation processes.

Conventional approaches to the treatment of type 2 diabetes (T2D) and obesity focus on the design of synthetic peptides that act as dual agonists or tri-agonists at G protein-coupled receptors (GPCRs), and that incorporate amino acid motifs of the hormones glucagon, glucagon-like peptide-1 (GLP-1), glucose-dependent insulinotropic hormone (GIP), and peptide YY (PYY). These synthetic peptides are under investigation owing to their predicted beneficial effects to control energy expenditure, appetite, and systemic glucose homeostasis. Glucagon, GLP-1, and GIP bind Family B GPCRs corresponding to the glucagon receptor (GluR), GLP-1 receptor (GLP-1R), and GIP receptor (GIPR), whereas the Family A neuropeptide Y₂ receptor (NPY2R) recognizes PYY.

The GluR, GLP-1R, and GIPR are expressed on multiple cell types including hepatocytes and adipocytes (glucagon), and pancreatic beta cells of the islets of Langerhans (GLP-1, GIP). Glucagon exerts catabolic actions to stimulate energy expenditure through glycogenolysis and lipolysis, whereas GLP-1 and GIP simulate insulin secretion so that levels of blood glucose are reduced. Whereas glucagon is secreted from islet alpha cells, GLP-1 and GIP are primarily secreted from enteroendocrine L-cells (GLP-1) and K-cells (GIP) that line the wall of the intestinal tract. Interestingly, L-cells co-secrete GLP-1 and PYY in response to nutrients present within the intestinal lumen. The PYY(1-36) precursor that is released is processed by dipeptidyl peptidase-4 (DPP-4) to generate circulating PYY(3-36) that crosses the blood-brain barrier so that it may suppress appetite by binding to NPY2R located on hypothalamic neurons. GLP-1 is also present within the solitary nucleus of the brainstem, and it too participates in the suppression of appetite by binding to a diffuse network of GLP-1 receptors located within the central nervous system.

To optimize dual agonist or tri-agonist peptides for therapeutic purposes, it is necessary to achieve “balanced agonism” in which simultaneous stimulation of multiple GPCRs is achieved across the desired concentration range. It is also necessary to identify the selectivity with which such peptides activate GPCRs, and in this regard it is necessary to identify potential non-conventional actions that allow them to exert off-target effects. As a result, there is a need in the art for an approach that can target the glucagon receptor (GluR), the glucagon-like peptide 1 receptor (GLP1-R) and neuropeptide Y2 receptor (NPY2-R) at the same time to more efficiently and effectively modulate glucoregulation and appetite regulation.

### BRIEF SUMMARY OF THE INVENTION

The present invention comprises the design for new monomeric peptide that can function at three receptors involved glucoregulation and appetite regulation. A peptide according to the present invention comprises a first amino acid sequence comprised of at least a portion of glucagon and a second amino acid sequence comprised of a portion of peptide YY (PYY₃₋₃₆) that is fused to a C-terminal of the first amino acid sequence. The first amino acid sequence may comprise the sequence HSQGTFTSDYSKYLDSRRAQDFVQWLMNT (SEQ. ID NO: 3). The protein sequence may comprise the sequence

(SEQ. ID NO: 1)
HSQGTFTSDYSKYLDSRRAQDFVQWLMNTRHYLNLVTRQRY-NH2.

The present invention also includes a method of simultaneously modulating glucoregulation and regulating appetite, comprising the step of administering a therapeutic amount of a peptide comprising a first amino acid sequence comprised of at least a portion of glucagon and a second amino acid sequence comprised of a portion of peptide YY (PYY₃₋₃₆) that is fused to a C-terminal of the first amino acid sequence. The first amino acid sequence may comprise HSQGTFTSDYSKYLDSRRAQDFVQWLMNT (SEQ. ID NO: 3). The protein sequence may comprise the sequence

(SEQ. ID NO: 1)
HSQGTFTSDYSKYLDSRRAQDFVQWLMNTRHYLNLVTRQRY-NH2.

Testing of SEQ. ID NO: 1, referred to as GGP817, confirmed that GGP817 targets the glucagon receptor (GluR), the glucagon-like peptide 1 receptor (GLP1-R) and neuropeptide Y2 receptor (NPY2-R).

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A is a graph of the agonistic dose response of GGP817 at the GLP-1 receptor (GLP-1R) in HEK cells expression the H188 viral FRET (485/535 nm) reporter;

FIG. 1B is a graph of the calculated EC50 (60.7 nM) of a plot of GGP817 concentration versus FRET (485/535 nm) ratio;

FIG. 2 is a graphs of the agonism of the NPY2 receptor by GGP817 in HEK cells expressing the H188 FRET reporter where EC50 is extrapolated to be ˜3 uM;

FIG. 3A is a graph of the agonistic dose response of GGP817 at the glugacon receptor (Gcg-R) in HEK cells expression the H188 viral FRET (485/535 nm) reporter;

FIG. 3B is a graph of the calculated EC50 (183.2 nM) from a plot of GGP817 concentration versus FRET (485/535 nm) ratio;

FIG. 4A is a graph of FRET data showing that LY2409021 blocks GGP817 agonist action at the GluR and the GLP-1R;

FIG. 4B is a box-and-whisker plot showing that LY2409021 blocks GGP817 agonist action at the GluR and the GLP-1R;

FIG. 4C is a dose-response plot summarizing findings in which GGP817 (EC₅₀ 183 nM) increased levels of cAMP in HEK293-GluR cells transduced with H188;

FIG. 5A is a first graph showing that LY2409021 (IC₅₀ 908 nM) blocked GGP817 (300 nM) action in HEK293-GluR cells transduced with H188;

FIG. 5B is a second graph showing that LY2409021 (IC₅₀ 908 nM) blocked GGP817 (300 nM) action in HEK293-GluR cells transduced with H188;

FIG. 5C is a third graph showing that LY2409021 (IC₅₀ 908 nM) blocked GGP817 (300 nM) action in HEK293-GluR cells transduced with H188;

FIG. 6A is a first graph showing GGP817 (EC₅₀ 61 nM) increased levels of cAMP in HEK293-GLP-1R cells transduced with H188;

FIG. 6B is a second graph showing GGP817 (EC₅₀ 61 nM) increased levels of cAMP in HEK293-GLP-1R cells transduced with H188;

FIG. 6C is a third graph showing GGP817 (EC₅₀ 61 nM) increased levels of cAMP in HEK293-GLP-1R cells transduced with H188;

FIG. 7A is a first graph showing LY2409021 (IC₅₀ 1.6 μM) blocked GGP817 (300 nM) action in HEK293-GLP-1R cells transduced with H188;

FIG. 7B is a second graph showing LY2409021 (IC₅₀ 1.6 μM) blocked GGP817 (300 nM) action in HEK293-GLP-1R cells transduced with H188;

FIG. 7C is a third graph showing LY2409021 (IC₅₀ 1.6 μM) blocked GGP817 (300 nM) action in HEK293-GLP-1R cells transduced with H188;

FIG. 8A is a graph of FRET data showing that GGP817 (IC₅₀ 19 nM) counteracted the cAMP-elevating action of adenosine (2 μM) in HEK293-H188-C24 cells transfected with NPY2R;

FIG. 8B is a box-and-whisker plot showing that GGP817 (IC₅₀ 19 nM) counteracted the cAMP-elevating action of adenosine (2 μM) in HEK293-H188-C24 cells transfected with NPY2R;

FIG. 8C is a dose-response plot showing that GGP817 (IC₅₀ 19 nM) counteracted the cAMP-elevating action of adenosine (2 μM) in HEK293-H188-C24 cells transfected with NPY2R;

FIG. 9A is a graph of findings obtained using HEK293-H188-C24 cells not treated with NPY2R antagonist BIIE0246 (2 μM), with the note that BIIE0246 blocked the cAMP-lowering action of GGP817 under conditions in which cells were also treated with adenosine (2 μM);

FIG. 9B is a graph of findings obtained using HEK293-H188-C24 cells treated with NPY2R antagonist BIIE0246 (2 μM);

FIG. 9C is a graph of a negative control demonstrating that GGP817 was without effect in HEK293-H188-C24 cells transfected with the empty vector (EV) and treated with adenosine

FIG. 10A is a graph of FRET data in which PYY(3-36) (IC50 18 nM) counteracted the cAMP-elevating action of adenosine (2 μM) in HEK293-H188-C24 cells transfected with NPY2R (a₁), (a₂), and (a₃) summarizing findings in which PYY(3-36) (IC₅₀ 18 nM) counteracted the cAMP-elevating action of adenosine (2 μM) in HEK293-H188-C24 cells transfected with NPY2R

FIG. 10B is a box-and-whisker plot in which PYY(3-36) (IC₅₀ 18 nM) counteracted the cAMP-elevating action of adenosine (2 μM) in HEK293-H188-C24 cells transfected with NPY2R; and

FIG. 10C is a dose-response plot in which PYY(3-36) (IC₅₀ 18 nM) counteracted the cAMP-elevating action of adenosine (2 μM) in HEK293-H188-C24 cells transfected with NPY2R.

### DETAILED DESCRIPTION OF THE INVENTION

Referring to the figures, wherein like numeral refer to like parts throughout, the present invention comprises a synthetic peptide that can simultaneously target the glucagon receptor (GluR), the glucagon-like peptide 1 receptor (GLP1-R) and neuropeptide Y2 receptor (NPY2-R). The peptide sequence may comprise

(SEQ. ID NO: 1)
HSQGTFTSDYSKYLDSRRAQDFVQWLMNTRHYLNLVTRQRY-NH2,

referred to as GGP817. GGP817 was designed from the naturally occurring native substrates for the glucagon receptor (GluR), the glucagon-like peptide 1 receptor (GLP1-R) and neuropeptide Y2 receptor (NPY2-R), e.g., PYY(3-36), IKPEAPGEDASPEELNRYYASLRHYLNLVTRQRY-NH2 (SEQ. ID. NO: 2), glucagon, HSQGTFTSDYSKYLDSRRAQDFVQWLMNT (SEQ. ID NO: 3) and GLP-1, HAEGTFTSDVSSYLEGQAAKEFIAWLVKG-NH2 (SEQ. ID NO: 4). GGP817 is a novel synthetic hybrid peptide that contains first sequence representing full-length glucagon to which a 12 amino acid C-terminal fragment of PYY(3-36) is fused at the C-terminal of the glucagon sequence.

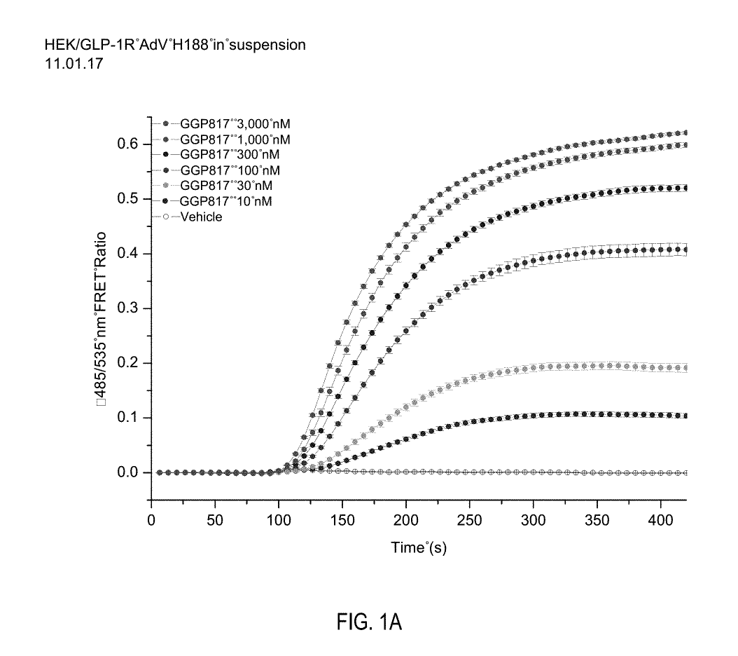

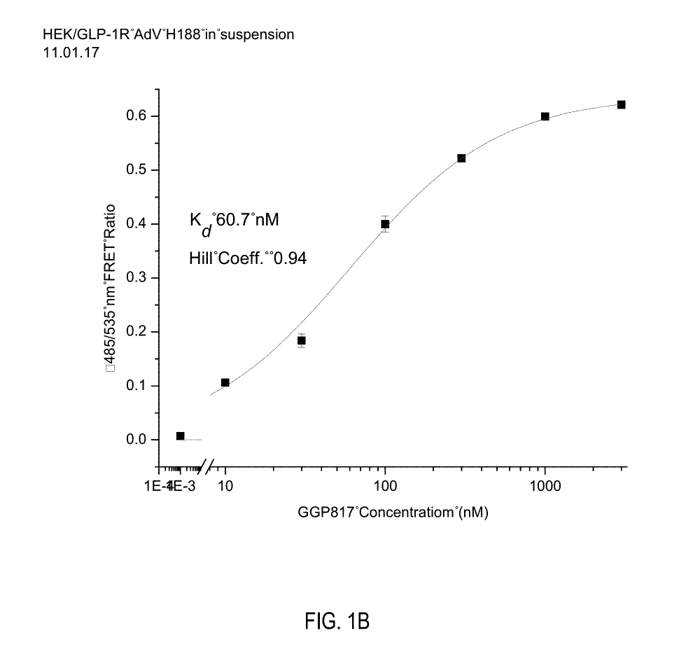

Referring to FIG. 1, an agonistic dose response of GGP817 at the GLP-1 receptor (GLP-1R) was demonstrated in HEK cells expressing the H188 viral FRET (485/535 nm) reporter show sufficient agonism and is supported by a calculated EC50 from a plot of GGP817 concentration versus FRET (485/535 nm) ratio.

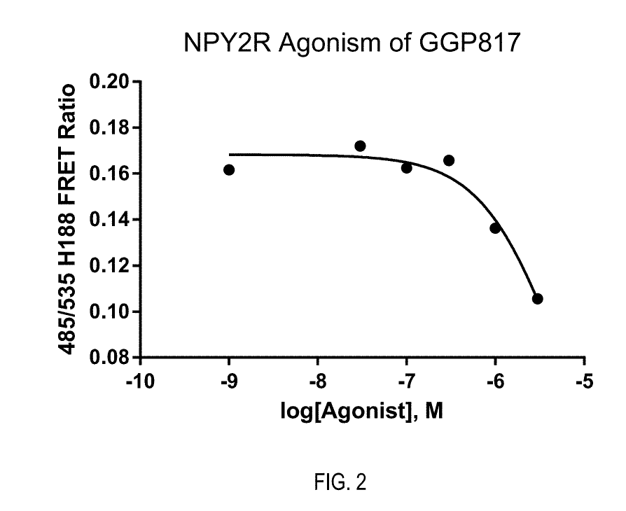

Referring to FIG. 2, agonism of the NPY2 receptor by GGP817 was also demonstrated in HEK cells expressing the H188 FRET reporter demonstrated an extrapolated EC50 of ˜3 uM.

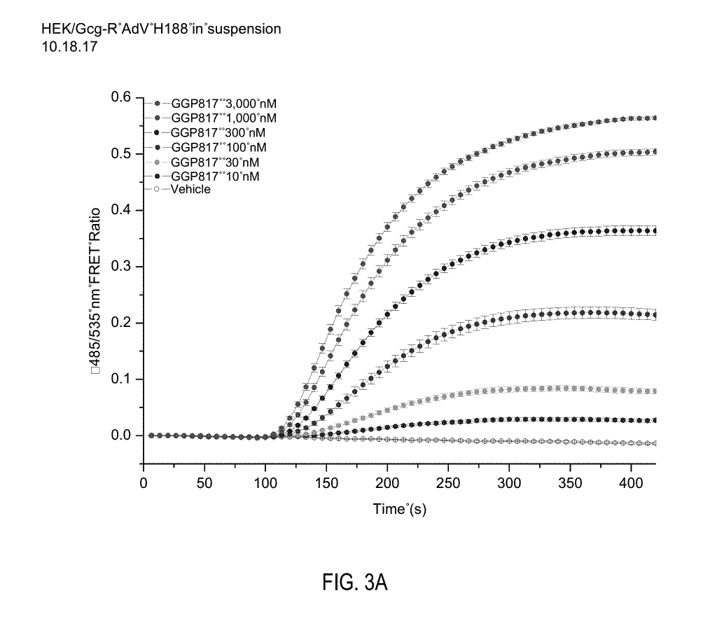

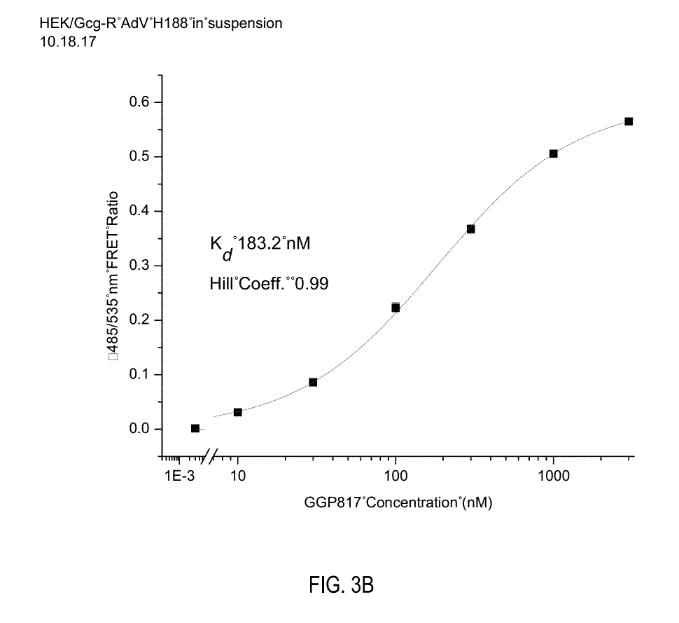

Referring to FIG. 3, an agonistic dose response of GGP817 at the glugacon receptor (Gcg-R) was demonstrated in HEK cells expression the H188 viral FRET (485/535 nm) reporter and is supported by a calculated EC50 (183.2 nM) from a plot of GGP817 concentration versus FRET (485/535 nm) ratio.

In addition to GGP817, several other synthetic peptides were designed using the same approach and are believed to also work as a triagonist with respect to the glucagon receptor (GluR), the glucagon-like peptide 1 receptor (GLP1-R) and neuropeptide Y2 receptor (NPY2-R). These synthetic peptides include Ac-HSQGTFTSDLSKQMEEEAVRLFIEWLKN RHYLNLVTRQRY-NH2 (SEQ. ID NO: 5), referred to as GGP002, Ac-HSQGTFT SDLSKQMEEEAVRLFIEWLKN RYYASLRHYLNLVTRQRY-NH2 (SEQ. ID NO: 6), referred to as GGP003, and Ac-HSQGTFTSDLSKQMEEEAVRLFIEWLKNGGPS RHYLNLVTRQRY-NH2 (SEQ. ID NO: 7), referred to as GGP004, and Ac-HSQGTFTSDLSKQMEEEAVRLFIEWLKNGGPS RYYASLRHYLNLVTRQRY-NH2 (SEQ. ID NO: 8), referred to as GGP005. The efficacy of each of these peptides may be confirmed by one of skill in the art using in vitro testing approaches shown in FIGS. 1 through 3. Following the approach of the present invention, another exemplary sequence constructed from fragments derived from the sequences of GLP1, PYY₃₋₃₆, and glucagon comprises HsQGT-FTSDY-SKYLD-EEAAR-LFIEW-LMNTR-YYASL-RHYLN-LVTRQ-RY (SEQ. ID NO: 9). In this example, several fragments from GLP1 have been incorporated along with glucagon residues and then fused to a PYY₃₋₃₆ at the C-terminus.

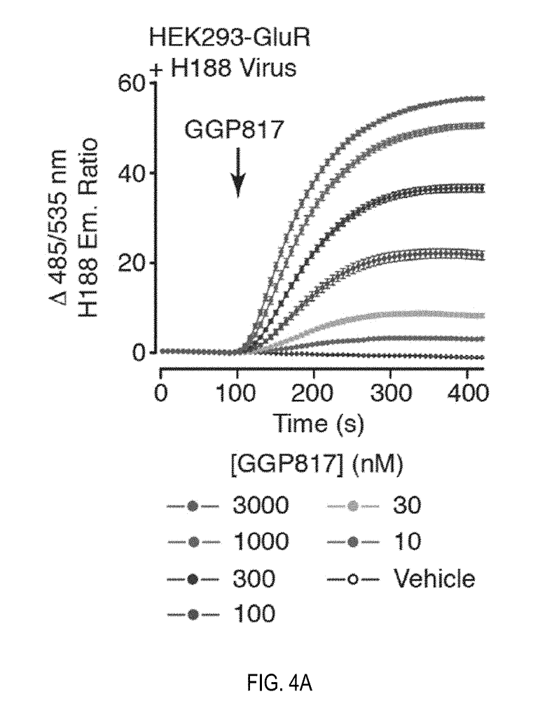

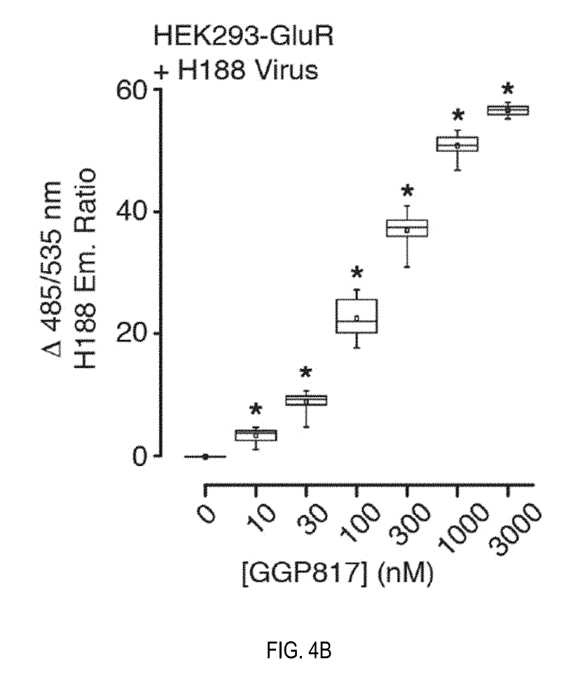

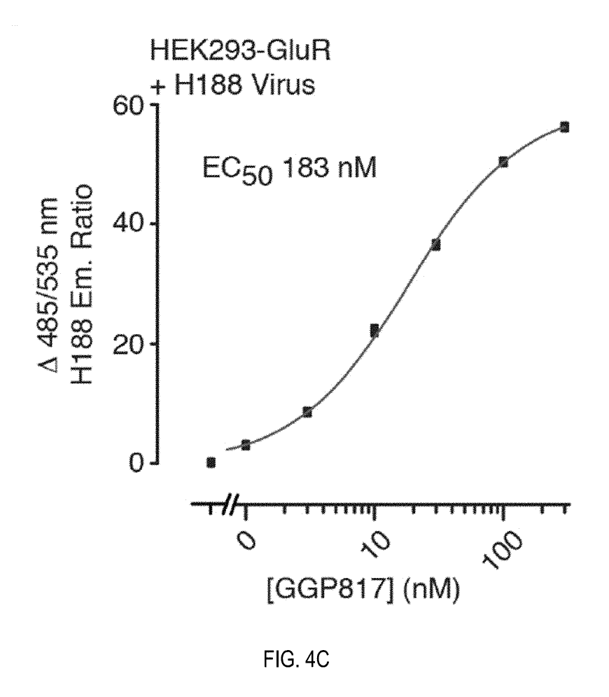

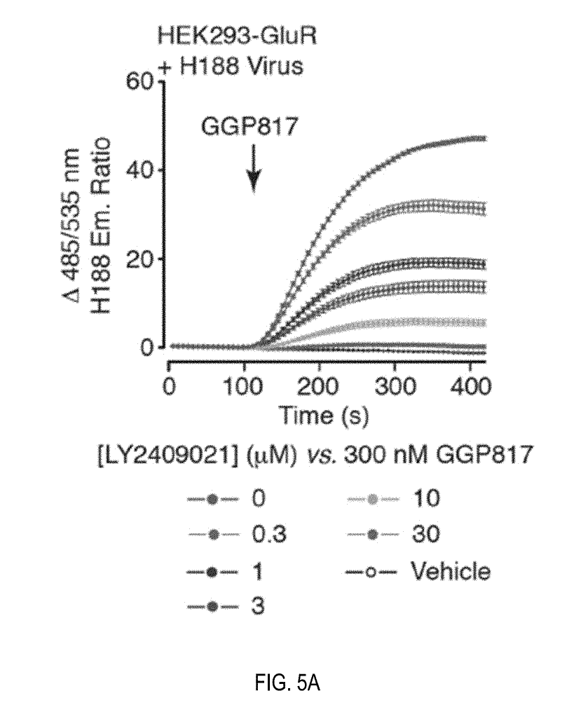

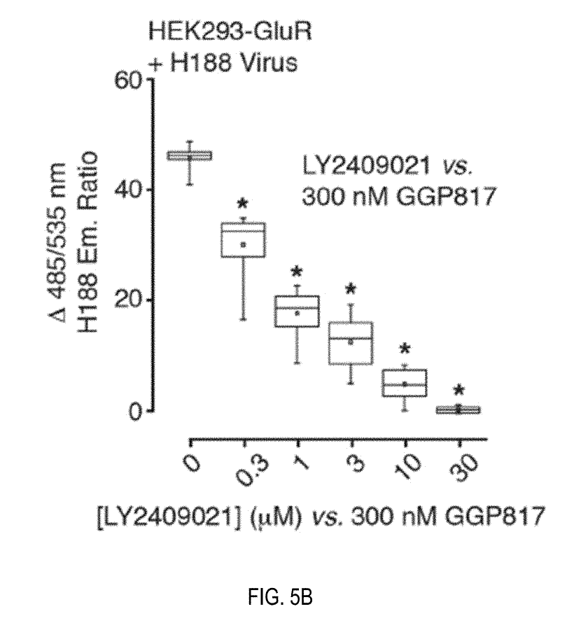

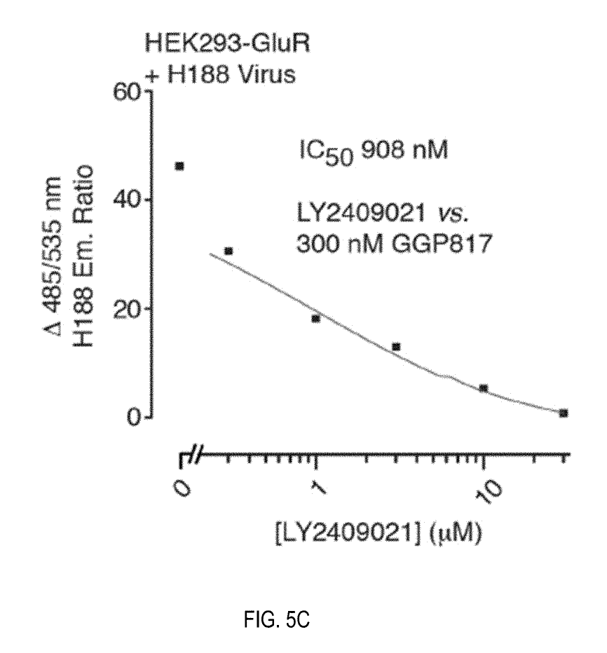

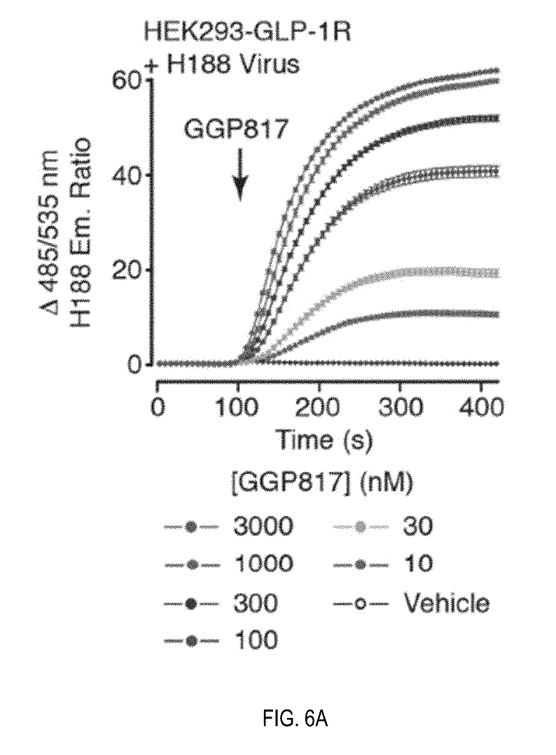

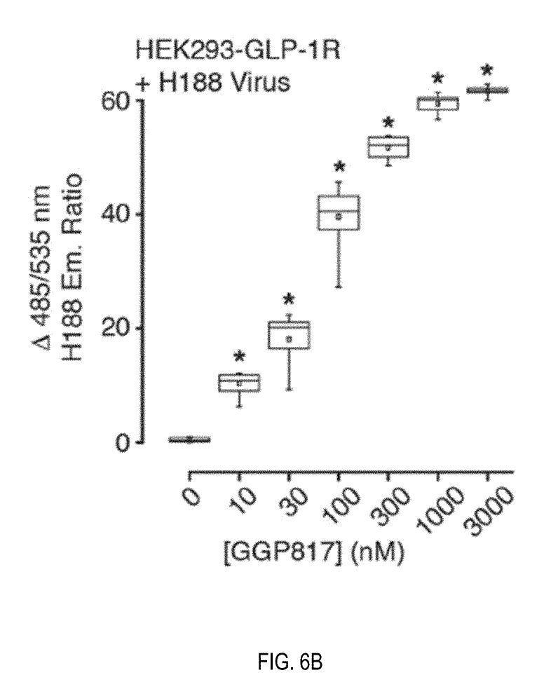

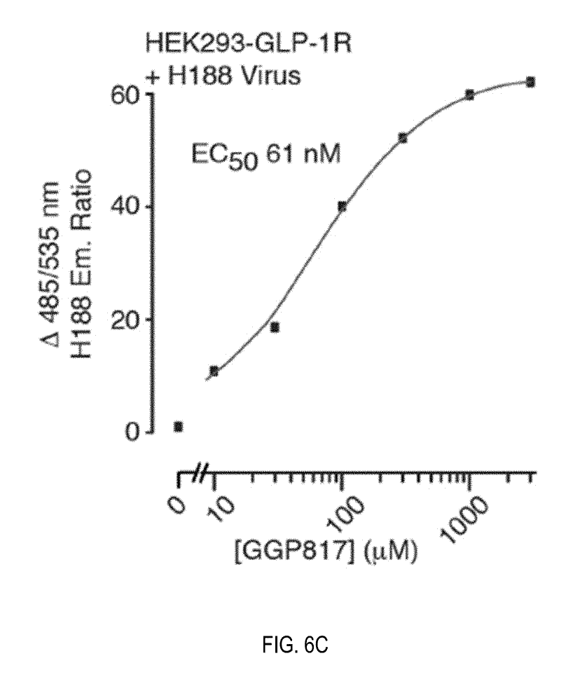

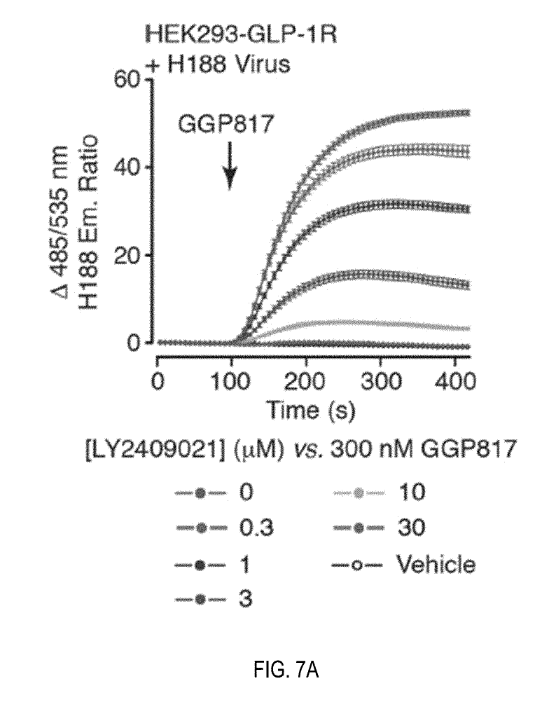

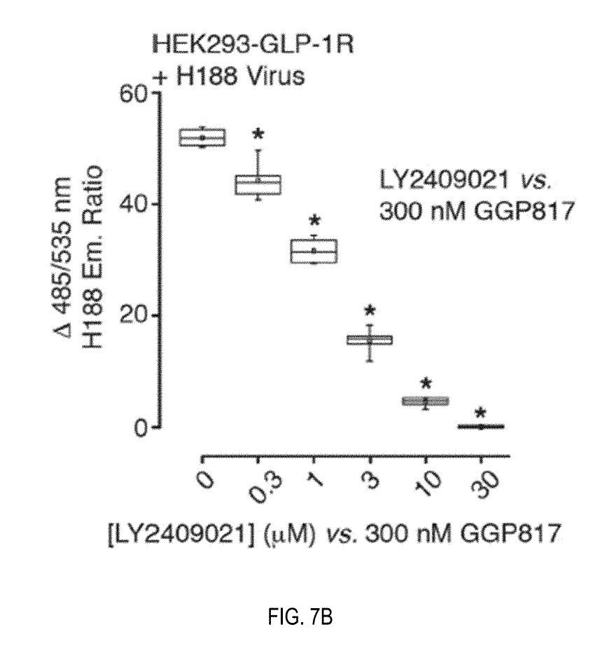

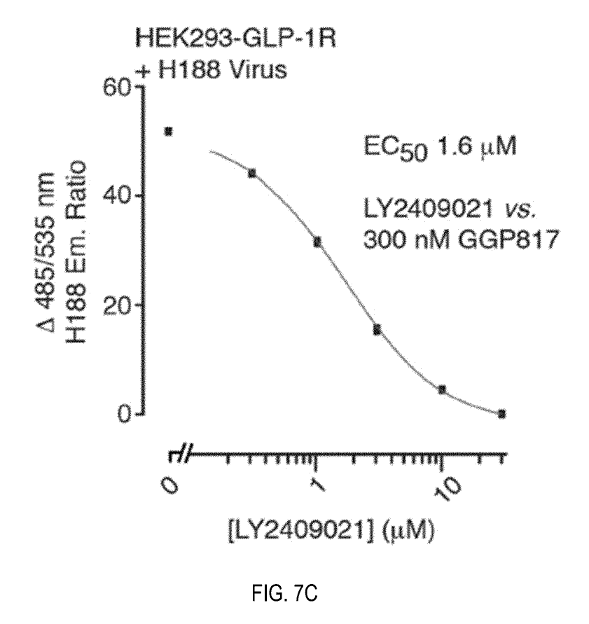

It is increasingly evident that hybrid peptides have the capacity to stimulate multiple GPCRs that participate in metabolic homeostasis. GGP817 was designed with the expectation that it might act as a tri-agonist since it incorporates amino acid residues found within glucagon, GLP-1, and PYY(3-36). For example, since glucagon is an agonist at the GluR, and also an agonist at the GLP-1R, GGP817 might possess tri-agonist properties in which it activates the GluR, GLP-1R, and NPY2R. When tested using HEK293-GluR cells, GGP817 exhibited agonist action at the GluR, see FIGS. 4A through 4C, and this effect was blocked by LY2409021 (a GluR allosteric inhibitor), see FIGS. 5A through 5C. However, GGP817 also exhibited agonist action at the GLP-1R, see FIGS. 6A through 6C, and this effect was also blocked by LY2409021, see FIGS. 7A through 7C. Note that when it was tested using GluR and GLP-1R expressing cells, GGP817 was less potent in comparison to the naturally occurring receptor ligands glucagon and GLP-1.

The EC₅₀ value for GGP817 agonist action at the GluR was 183 nM, see FIG. 4C, whereas it was 61 nM for the GLP-1R, see FIG. 6C. Still, when GGP817 was tested at a saturating concentration (3,000 nM), it acted as a full agonist at both the GluR and the GLP-1R. This was established by monitoring its ability to stimulate a 60% maximal ΔFRET in the cAMP assay. Of interest, the antagonist potency of LY2409021 at the GluR and GLP-1R in assays using GGP817 resembled that which was observed when testing LY2409021 in assays using glucagon as a stimulus for GluR or GLP-1R activation (c.f., FIG. 2b₃, 2d₃, 4b₃, 4d₃). For example, when comparing antagonist actions of LY2409021 to block GGP817 agonist action at the GluR (FIG. 4b₃), or GLP-1 agonist action at the GLP-1R (FIG. 4d₃), the IC₅₀ values were 908 nM and 1.6 μM, respectively.

Since GGP817 contains a C-terminal fragment of PYY(3-36) fused to glucagon, it was tested to determine if it might bind to NPY2R to activate Gᵢ proteins, thereby reducing levels of cAMP. To test this, HEK293-H188-C24 cells were transfected with human NPY2R (42), so that cAMP-lowering actions of GGP817 and NPY2R agonist PYY(3-36) could be compared. Since HEK293-H188-C24 cells have low basal adenylyl cyclase activity and low basal levels of cAMP (24), adenosine (acting at endogenous A2_{B} receptors) or forskolin (acting at adenylyl cyclase) was used to initially enhance cyclase activity, thereby raising levels of cAMP prior to inhibitory agonist treatment. In this manner, an NPY2R mediated counter regulatory action of GGP817 to lower levels of cAMP could be evaluated.

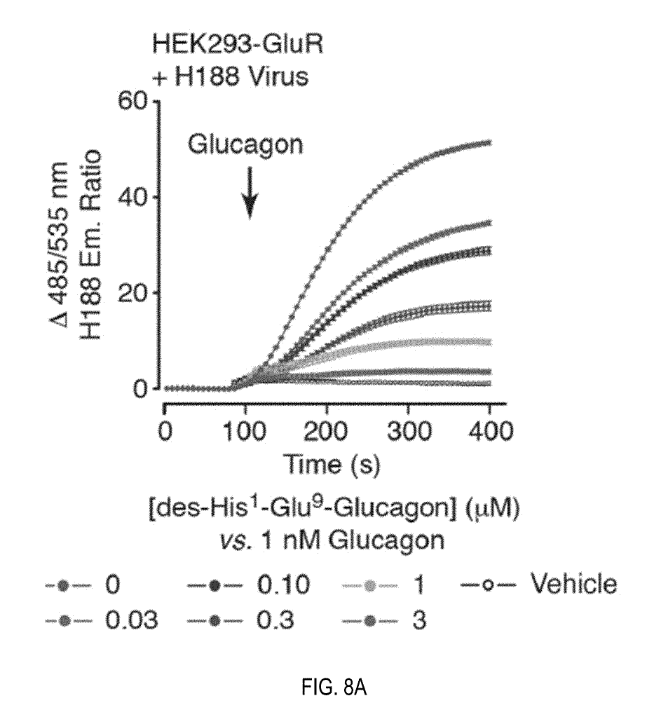

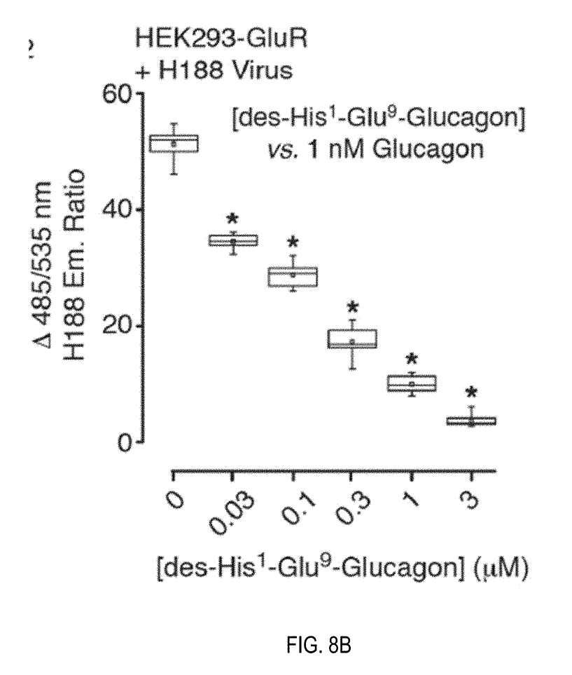

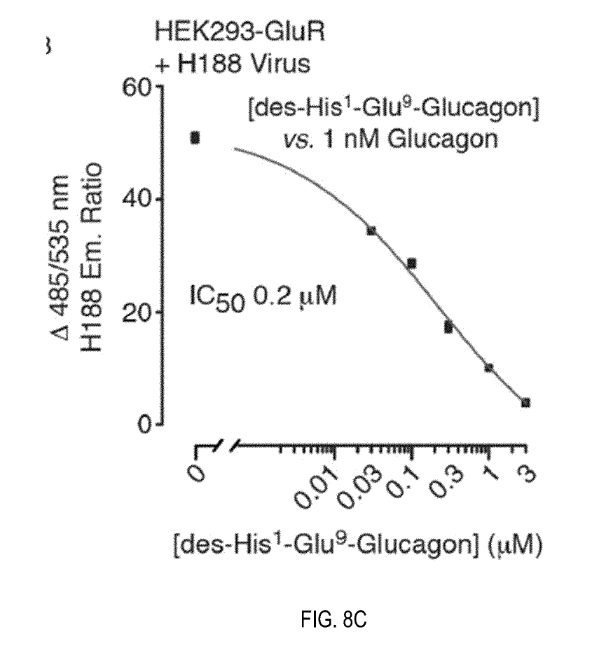

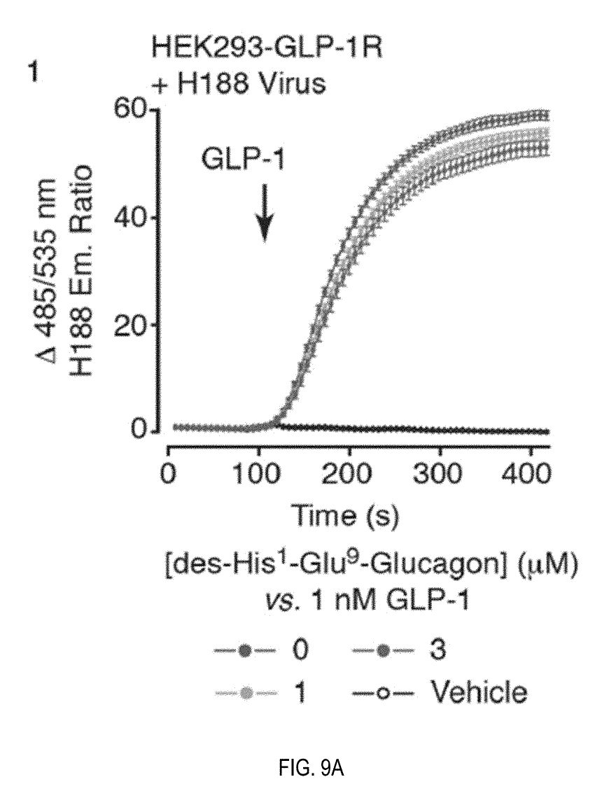

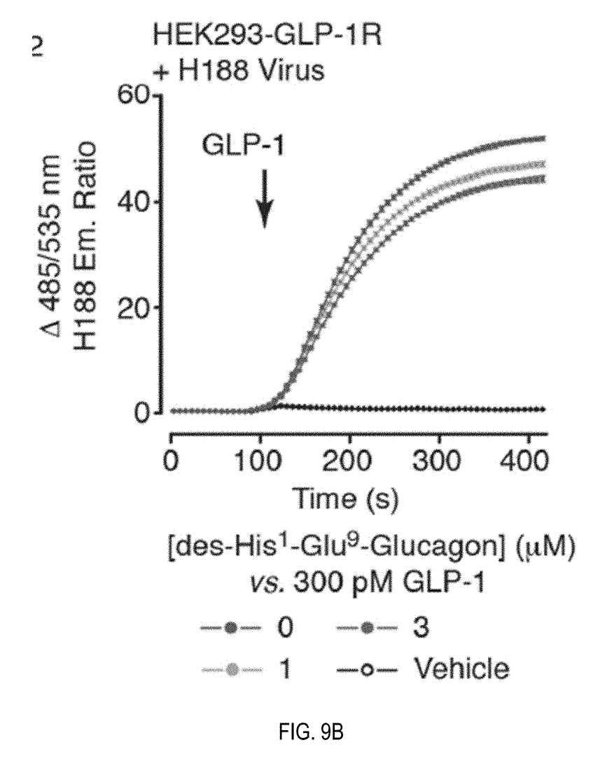

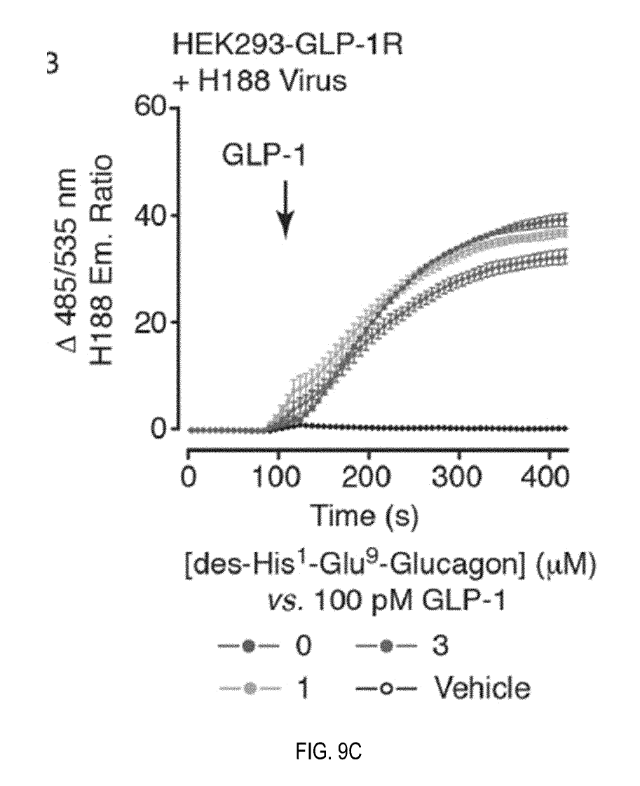

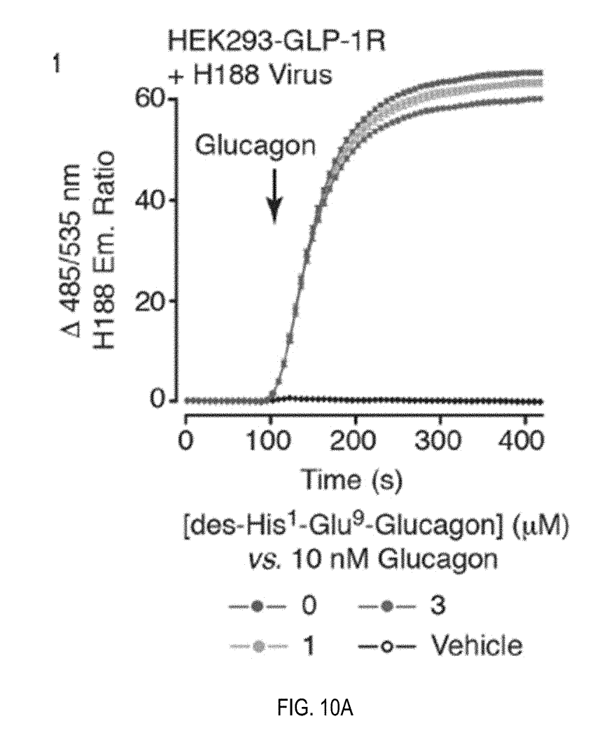

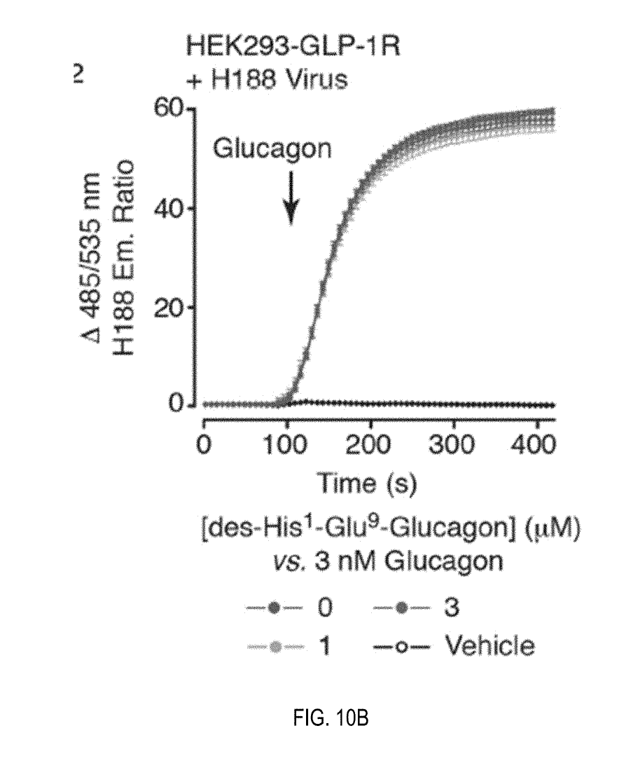

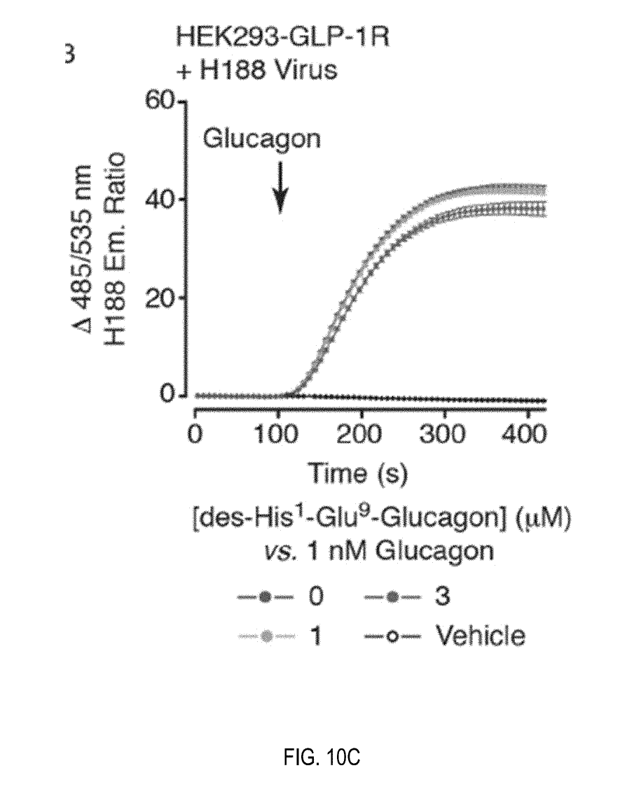

When HEK293-H188-C24 cells expressing NPY2R were stimulated with adenosine (2 μM), levels of cAMP rose, and this effect was counteracted by GGP817 (10-1,000 nM), see FIGS. 8A through 8C. By comparing cells not treated or treated with NPY2R antagonist BIIE0246 (500 nM), it was established that the cAMP-lowering action of GGP817 was NPY2R mediated, see FIGS. 9A and 9B. Importantly, GGP817 was without effect in HEK293-H188-C24 cells transfected with an empty vector (EV) that served as a negative control, see FIG. 9C. NPY2R agonist PYY(3-36) replicated the cAMP-lowering action of GGP817 across similar dose ranges, see FIGS. 10A through 10C. In fact, the IC50 values for GGP817 (19 nM) and PYY(3-36) (18 nM) agonist actions were nearly identical. To obtain independent confirmation of these findings using adenosine, it was demonstrated that GGP817 counteracted the cAMP-elevating action of forskolin in assays using HEK293-H188-C24 cells transfected with NPY2R, but not the EV. Finally, it was demonstrated that in an assay using forskolin instead of adenosine, the cAMP-lowering action of GGP817 was reproduced by PYY(3-36).

These tests demonstrated that GGP817 exhibits tri-agonist properties so that it acts at the GluR and GLP-1R to stimulate cAMP production, while it also acts at NPY2R to inhibit cAMP production.

What is claimed is:

1. A peptide for simultaneously targeting the glucagon receptor (GluR), the glucagon-like peptide 1 receptor (GLP1-R) and neuropeptide Y2 receptor (NPY2-R), comprising:
a first amino acid sequence comprised of at least a portion of glucagon; and
a second amino acid sequence comprised of a portion of peptide YY (PYY₃₋₃₆) that is fused to a C-terminal of the first amino acid sequence.

2. The peptide of claim 1, wherein the second amino acid sequence has twelve amino acids.

3. The peptide of claim 2, wherein the first amino acid sequence comprises
(SEQ. ID NO: 3)
HSQGTFTSDYSKYLDSRRAQDFVQWLMNT

4. The peptide of claim 3, wherein peptide comprises the sequence
(SEQ. ID NO: 1)
HSQGTFTSDYSKYLDSRRAQDFVQWLMNTRHYLNLVTRQRY-NH2.

5. The peptide of claim 1, wherein the peptide exhibits agonist action at GluR.

6. The peptide of claim 1, wherein the peptide exhibits agonist action at GLP-1R.

7. The peptide of claim 1, wherein the peptide exhibits agonist action at NPY2-R.

8. A method of simultaneously modulating glucoregulation and regulating appetite in a subject, comprising the step of administering to the subject a therapeutic amount of a peptide comprising a first amino acid sequence comprised of at least a portion of glucagon and a second amino acid sequence comprised of a portion of peptide YY (PYY₃₋₃₆) that is fused to a C-terminal of the first amino acid sequence.

9. The method of claim 8, wherein the second amino acid sequence has twelve amino acids.

10. The method of claim 9, wherein the first amino acid sequence comprises
(SEQ. ID NO: 3)
HSQGTFTSDYSKYLDSRRAQDFVQWLMNT

11. The method of claim 10, wherein peptide comprises the sequence
(SEQ. ID NO: 1)
HSQGTFTSDYSKYLDSRRAQDFVQWLMNTRHYLNLVTRQRY-NH2.

12. The method of claim 8, wherein administration of the peptide results in agonist action at GluR.

13. The method of claim 8, wherein administration of the peptide results in agonist action at GLP-1R.

14. The method of claim 8, wherein administration of the peptide results in agonist action at NPY2-R.
